(19) United States
(12) Patent Application Publication
(10) Pub. No.: US 2007/0237287 A1
(43) Pub. Date: Oct. 11, 2007

(54) CT SCANNER WITH AUTOMATIC DETERMINATION OF VOLUME OF INTEREST

(76) Inventors: Predrag Sukovic, Birmingham, MI (US); Joseph Webster Stayman, Ann Arbor, MI (US); David Phillipe Sarment, Ann Arbor, MI (US); Neal Clinthorne, Ann Arbor, MI (US)

Correspondence Address: CARLSON, GASKEY & OLDS, P.C., 400 WEST MAPLE ROAD, SUITE 350, BIRMINGHAM, MI, 48009, US

(21) Appl. No.: 11/692,380

(22) Filed: Mar. 28, 2007

Related U.S. Application Data
(60) Provisional application No. 60/786,638, filed on Mar. 28, 2006.
(60) Provisional application No. 60/851,196, filed on Oct. 12, 2006.

(51) Int. Cl. H05G 1/60; A61B 6/00; G01N 23/00; G21K 1/12

(52) U.S. Cl. 378/4

(57) ABSTRACT

A CT scanner automatically determines a volume of change based upon anatomical changes in a patient. During surgery, the CT scanner takes a sufficient number of two-dimensional initial images using a full field of view. The CT scanner compares the initial images to pre-operative data. Based upon the comparison, the CT scanner automatically determines the volume of change plus some margin to define a volume of interest. The CT scanner then collimates an x-ray source to perform an intra-operative updated CT scan of the volume of interest. The CT scanner updates the pre-operative data with the data from the intra-operative updated CT scan of the volume of interest to form a fully updated three-dimensional CT image. The initial images and the pre-operative data can be taken at a lower resolution than the intra-operative updated CT scan of the volume of interest to reduce the x-ray exposure of the patient.

### REFERENCE TO RELATED APPLICATIONS

This application claims priority to U.S. Provisional Application Nos. 60/786,638 filed Mar. 28, 2006 and 60/851,196 filed Oct. 12, 2006.

### BACKGROUND OF THE INVENTION

The present invention relates generally to a surgical imaging system including a CT scanner that automatically determines a volume of interest of a patient.

It is sometimes desirable to be able to take a CT scan of a patient during surgery. For example, a surgeon may want to check the progress of the surgery (e.g., to determine whether a problem has been completely corrected or whether a tumor has been completely removed, etc).

For image-guided surgery, it is sometimes desirable to periodically update a pre-operative CT scan of the patient. The relevant volume of the patient may have shifted between the time the pre-operative image was taken and the time of surgery. This is especially true once surgery has begun. For example, in cranial surgery, a shape of an intracranial cavity changes as the surgeon gains access. Changes in the pre-operative image and the actual surgical subject introduce variations into the surgical process. In matters like intracranial surgery, the tolerance for variations is low, thus even small changes between the image and actual subject may cause problems and make the surgery less effective.

To solve this problem, a new, partial CT scan may be taken during surgery to update the previously received information. It is known that a baseline, pre-operative CT scan can be updated with a partial CT scan of a volume of interest in which an x-ray source is collimated to scan only the volume of interest. The partial CT scan is used in conjunction with the pre-operative CT scan (which includes volumes that have presumably not changed) to obtain a full CT image.

However, selecting the volume of interest can be time-consuming if the surgeon is required to find the volume of interest on the pre-operative CT scan. Also, if the patient and/or the CT scanner have moved, the relative locations of the CT scanner and patient must be determined and registered with the image guided surgical system before the location of the volume of interest can be determined.

### SUMMARY OF THE INVENTION

A CT scanner automatically determines a volume of change of a patient based upon anatomical changes that can be determined by the CT scanner in one or more (but significantly less than a full set) of frames. During surgery, when an updated CT scan is requested, the CT scanner begins performing a scan of a patient using a full field of view. The CT scanner takes a series of two-dimensional initial images (or “frames”) of the patient from a plurality of angularly spaced positions about the patient. When a sufficient number of initial images have been obtained, the CT scanner first registers its location relative to the patient and the previous CT images based upon the initial images. In other words, based on the initial images, the CT scanner determines its position relative to the current position of the patient and the current position of the patient relative to the previous CT scan(s).

When operating in an automatic mode, the CT scanner then compares the initial images to a previous CT image (either a pre-operative CT scan, a previously-updated CT scan or generic data). Based upon the comparison, the CT scanner determines where changes to the anatomy have occurred (e.g., because of the surgery so far). The volume where changes have occurred, plus some defined margin, becomes the volume of interest. The CT scanner then collimates an x-ray source to perform an intra-operative updated CT scan of only the volume of interest. The CT scanner updates the previous CT scan(s) with the new images of the volume of interest from the intra-operative updated CT scan to create a fully updated CT image, reducing x-ray exposure of the patient.

Alternatively, the surgeon can manually select the volume of interest on a previously-stored CT image using a computer user interface (graphic user interface or voice-activated, etc). In this example, the volume of change has been automatically determined by the CT scanner. The CT scanner then determines its location relative to the patient based upon the initial images. Then, with its location registered relative to the patient, the CT scanner collimates the x-ray source and focuses on the manually-defined volume of interest and completes the intra-operative updated CT scan of the volume of interest.

Preferably, the CT scanner provides a user interface that offers the surgeon the option of performing either the fully automatic volume of interest determination update scan or a surgeon-selected volume of interest determination update scan with automatic registration of the volume of change by the CT scanner.

Alternately, the pre-operative data and the initial images can be taken using a lower resolution and optionally, a low x-ray dosage. The CT scanner generates lower resolution images by downsampling the information from a detector. After the volume of interest is determined, the x-ray source is collimated and a CT scan of the volume of interest is taken at a higher resolution than the pre-operative data and the initial images.

### BRIEF DESCRIPTION OF THE DRAWINGS

FIG. 1 schematically illustrates a first embodiment CT scanner;

FIG. 2 illustrates the CT scanner of FIG. 1 with a part of a person received in the CT scanner;

FIG. 3 illustrates a second embodiment of the CT scanner;

FIG. 4 illustrates a computer employed with the CT scanner;

FIG. 5 illustrates a first full field of view of a two-dimensional CT image;

FIG. 6 illustrates a second full field of view of a two-dimensional CT image; and

FIG. 7 illustrates a collimated field of view of a two-dimensional CT image that focuses on a volume of interest.

### DETAILED DESCRIPTION OF THE PREFERRED EMBODIMENT

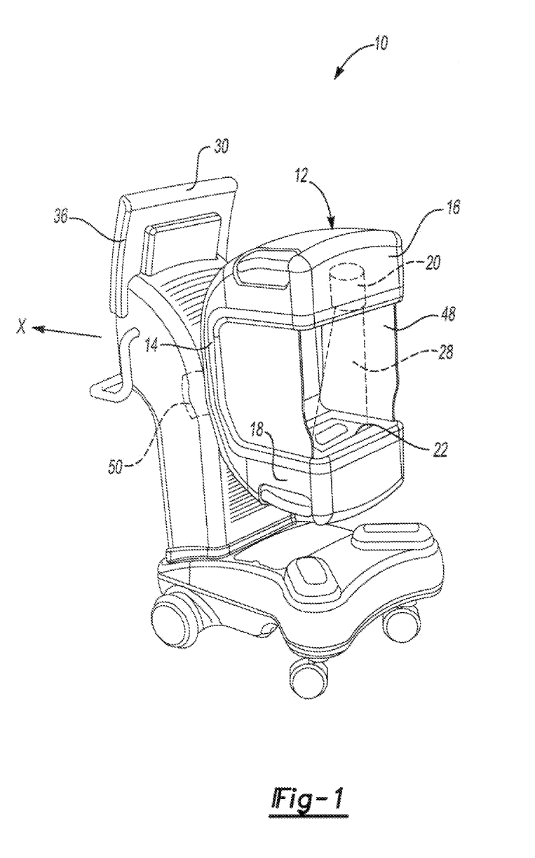

FIG. 1 illustrates an intra-operative CT scanner 10 of a surgical imaging system of the present invention including a gantry 12 that supports and houses components of the CT scanner 10. Suitable CT scanners 10 are known. In one example, the gantry 12 includes a cross-bar section 14, and a first arm 16 and a second arm 18 each extend substantially perpendicularly from opposing ends of the cross-bar section 14 to form the c-shaped gantry 12. The first arm 16 houses an x-ray source 20 that generate x-rays 28. In one example, the x-ray source 20 is a cone-beam x-ray source. The second arm 18 houses a complementary flat-panel detector 22 spaced apart from the x-ray source 20. The x-rays 28 are directed toward the detector 22 which includes a converter (not shown) that converts the x-rays 28 from the x-ray source 20 to visible light and an array of photodetectors behind the converter to create an image. As the gantry 12 rotates about the patient P, the detector 22 takes a plurality of x-ray images at a plurality of rotational positions. Various configurations and types of x-ray sources 20 and detectors 22 can be utilized, and the invention is largely independent of the specific technology used for the CT scanner 10.

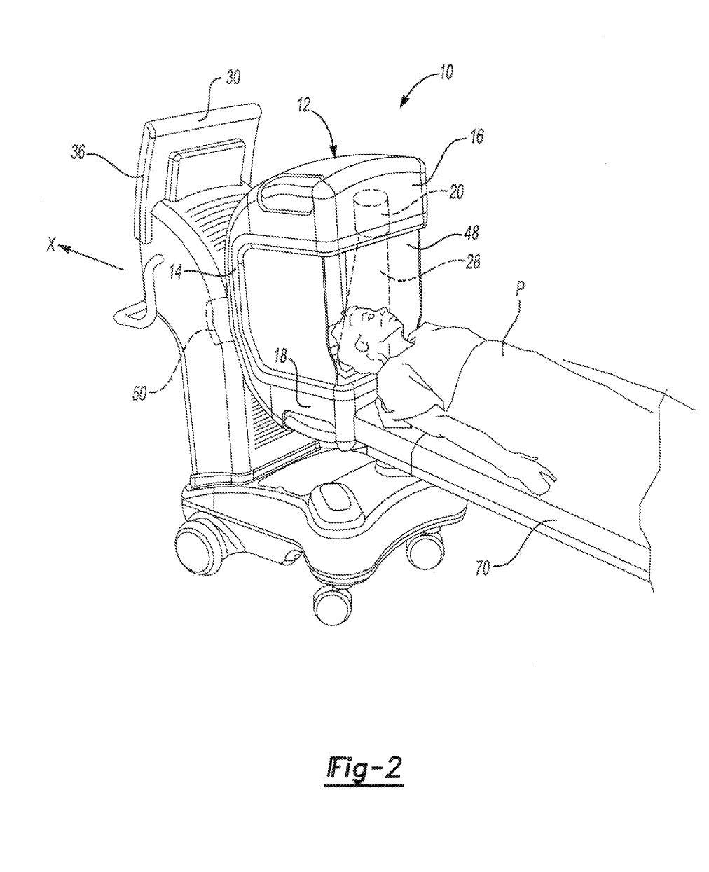

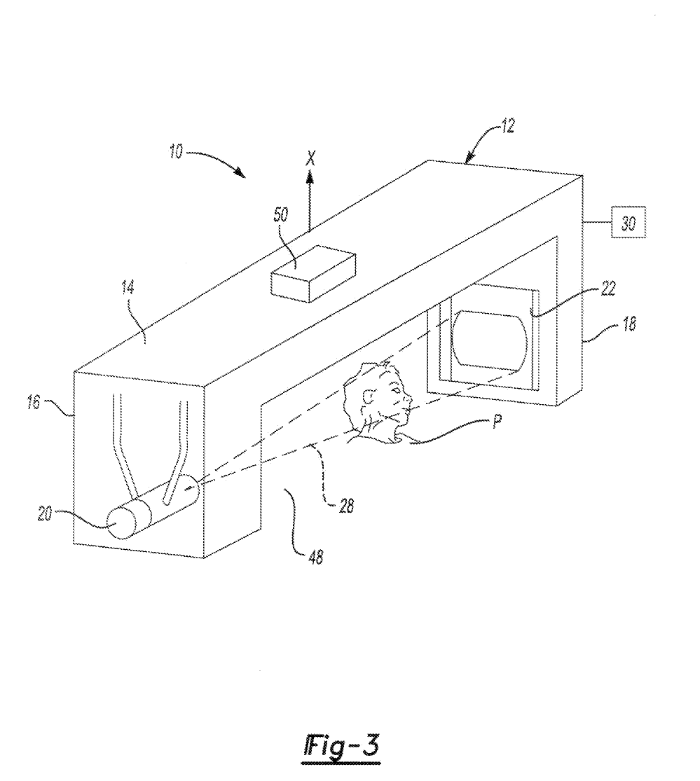

FIG. 2 illustrates the CT scanner 10 with a part of the patient P received in a space 48 between the first arm 16 and the second arm 18. A motor 50 rotates the gantry 12 about an axis of rotation X to obtain a plurality of x-ray images of the patient P at the plurality of rotational positions. The axis of rotation X is substantially centered within the gantry 12 and positioned between the x-ray source 20 and the detector 22. The gantry 12 can be rotated approximately slightly more than 360 degrees about the axis of rotation X. In one example, as shown in FIGS. 1 and 2, the axis of rotation X is substantially horizontal. In this example, the patient P is typically lying down on a table 70. Alternatively, as shown in FIG. 3, the axis of rotation X is substantially vertical. Typically, in this example, the patient P is sitting upright.

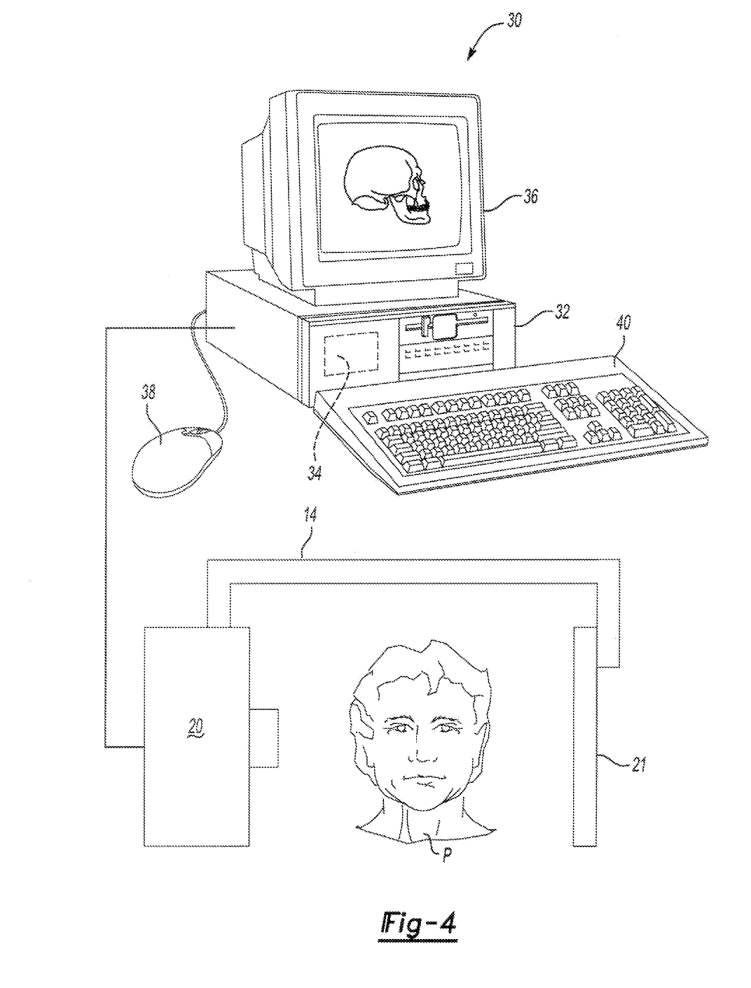

As shown schematically in FIG. 4, the CT scanner 10 further includes a computer 30 having a microprocessor or CPU 32, a storage 34 (memory, hard drive, optical, and/or magnetic, etc), a display 36, a mouse 38, a keyboard 40 and other hardware and software for performing the functions described herein. The computer 30 powers and controls the x-ray source 20 and the motor 50. The plurality of x-ray images taken by the detector 22 are sent to the computer 30. The computer 30 generates a three-dimensional CT image from the plurality of x-ray images utilizing any known techniques and algorithms. The three-dimensional CT image is stored on the storage 34 of the computer 30 and can be displayed on the display 36 for viewing.

Returning to FIG. 1, prior to surgery, a full pre-operative scan (CT or MRI) of the patient P is performed and stored on the computer 30. As an alternative, for some types of surgery, it is possible that some generic data (i.e., data not specifically from the present patient P) describing the area surrounding a volume of interest 59 may be sufficient. The pre-operative data may be a complete three-dimensional CT image or model or a partial three-dimensional CT image or model of the area surrounding the volume of interest 59.

During surgery, the CT scanner 10 takes intra-operative CT scans of the volume of interest 59 within the patient P so that the surgeon (or a dentist) can determine the current progress of the surgery (e.g., has a tumor been completely removed or a sinus cavity been completely repaired?) The CT scanner 10 only performs a complete CT scan of the volume of interest 59, which is the volume (or volumes) where the surgeon is working. The volume of interest 59 is defined as the volume of change 57 in the anatomy of the patient P as detected, plus some margin.

The computer 30 uses the pre-operative data surrounding the volume of interest 59 in conjunction with the new information from the intra-operative CT scans to create a fully updated three-dimensional CT image. Therefore, a new, full intra-operative CT scan is not required to form a CT image of the volume of interest 59. The smaller scan of the volume of interest 59 also reduces the dosage of x-rays experienced by the patient P.

When a surgeon determines that an updated CT image is needed, a fully automatic updated CT scan or a manually designated updated CT scan of the volume of interest 59 can be requested. The user interface offers the surgeon the option of performing either the fully automatic volume of interest determination update scan or an updated CT scan of a manually selected volume of interest, both with automatic registration by the CT scanner 10. The request can be made by either using a graphical or voice-activated user interface on the computer 30.

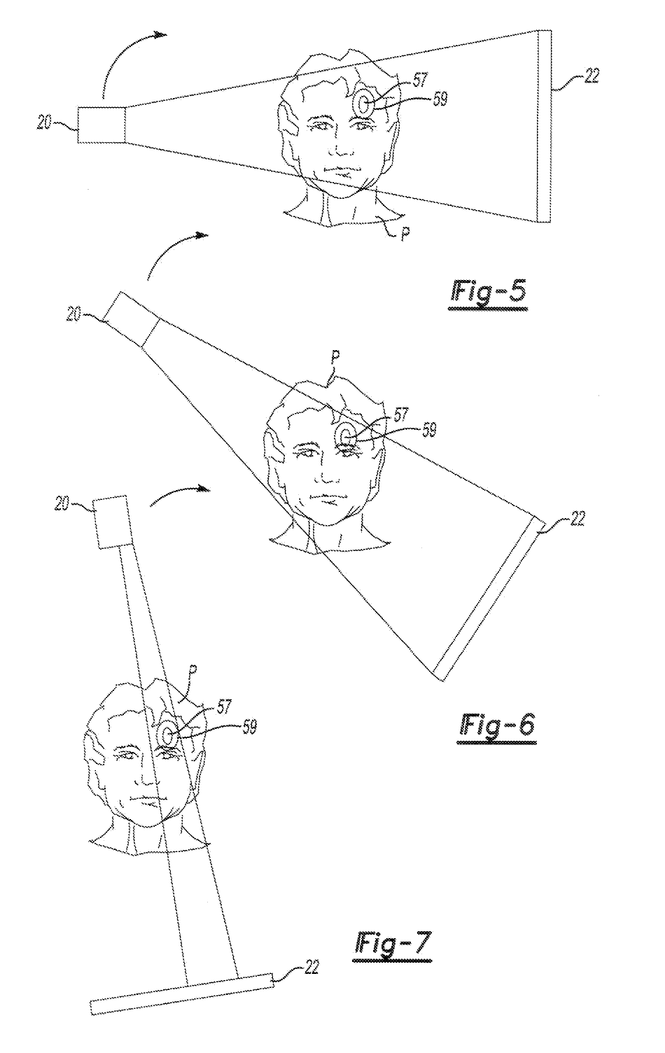

When a fully automatic updated CT scan is requested, the CT scanner 10 takes a series of full field of view, two-dimensional CT images (initial images) from a plurality of angularly separated positions about the patient P (as shown in FIGS. 5 and 6, although more images could be used). The plurality of positions may be the same angularly-spaced positions that would be used in a full CT scan or may be separated by much larger angles (so that fewer positions could be used). For an illustrative example only, between two and ten initial images could be taken over approximately 45 degrees.

The initial images provide two primary purposes: 1) to determine the position of the CT scanner 10 relative to the patient P (who may have been moved during the surgery); and 2) to determine the location of the volume of interest 59. Additionally, the initial images (or portions of them) may be used to perform the update.

Based upon the initial images, the CT scanner 10 first registers its location relative to the patient P (who may have moved during surgery) and the previous CT scans. This can be done by locating and orienting a known structure in this part of the patient's P anatomy (e.g., part of the skull) in the initial images. The CT scanner 10 then compares the initial images to a previous CT image (the pre-operative scan, a previously-updated scan or generic data). Based upon the comparison, the CT scanner 10 determines where changes to the patient's P anatomy have occurred (e.g., because of the surgery so far). The volume of change 57, plus some defined margin, becomes the volume of interest 59.

The CT scanner 10 then collimates the x-ray source 20 and takes a plurality of images at a plurality of angularly-spaced positions to perform the intra-operative updated CT scan of the volume of interest 59, as shown in FIG. 7. After collimating, the CT scanner 10 takes images at the regularly spaced intervals for the remainder of the CT scan (approximately 180 to 360 degrees).

Alternatively, the surgeon can select “manual designation” to manually select the volume of interest 59 on the previously-stored CT image using software on the computer 30. After the CT scanner 10 locates the volume of change 57 and displays it to the surgeon, the surgeon can circle the volume of interest 59 on a three dimensional CT image on the computer 30 by using the mouse 38 or the keyboard 40. That is, the CT scanner 10 determines the volume of change 57, and the surgeon selects the size of the volume of interest 59. The CT scanner 10 then determines its location relative to the patient P based upon the initial images. Then, with its location registered relative to the patient P, the CT scanner 10 focuses in on the volume of interest 59 and completes the updated CT scan.

If more than one volume of change 57 is detected in the initial images, the CT scanner 10 could present the surgeon with the option of choosing one or more of the volumes of change 57 as the volume of interest(s) 59. If more than one volume of interest 59 is selected, the CT scanner 10 could then collimate to obtain images for each of the volumes of interest 59 in alternating frames as the CT scanner 10 rotates around the patient P. The CT scanner 10 could also perform multiple rotations about the patient P (or multiple 180 degree scans, or anywhere between 180 and 360 degrees for each scan).

The CT scanner 10 then automatically (i.e., without further prompting or input) displays the volume of interest 59 on the display 36. If more than one volume of interest 59 was selected, the CT scanner 10 marks the locations of the volumes of interest 59 so the surgeon can easily toggle or scroll between the volumes of interest 59.

The feature of registering the location of the CT scanner 10 relative to the current patient location could be used independently of the automatic determination of the volume of interest 59 feature, and vice versa.

The intra-operative updated CT scan of the volume of interest 59 could be performed at a different resolution than the pre-operative scan or data and the initial images. The pre-operative data is used only for background information and calculations required in creating a new image and has lesser importance in the new image than does the intra-operative data. The pre-operative CT scan may use a lower resolution and optionally, a low x-ray dosage, than the intra-operative scan, resulting in a safer pre-operative scan for the patient P and a cost savings in obtaining the pre-operative scan. The pre-operative CT scan is also a smaller computer file and therefore takes up less space and uses less processing power.

When desired, the CT scanner 10 generates lower resolution images by downsampling the information from the detector 22. Downsampling includes any of several methods for reducing the resolution of the information from the detector 22. In one example, a certain percentage of the pixels are ignored. For example, every other pixel or every third pixel, etc. is sampled Another way of downsampling is to average together the signal from adjacent pixels, such as an adjacent pair or a small array of four or more pixels, and then to treat it as a single pixel of information. Information from adjacent pixels can be statistically combined in many different ways besides averaging. The amount of downsampling (or not downsampling at all) can be varied by the CT scanner 10. The amount of downsampling (the resolution of the image) can even be varied within an image, as controlled by the computer 30, such that selected volumes of the image are at a higher resolution than the remainder of the image. In this manner, for example, a volume of interest 59 within the patient P can be recorded at a high resolution, without unnecessarily increasing the image file size for the entire image. Varying the resolution of the image can be used in several different ways, as will be explained below.

For example, the CT scanner 10 takes a plurality of downsampled (low resolution) initial images at the full field of view to determine the volume of interest 59. After the volume of interest 59 is located on the downsampled initial images, the x-ray source 20 is collimated, and a CT scan of the volume of interest 59 is taken at a higher resolution (or higher dosage) than the pre-operative images and the initial images.

Alternatively, with the x-ray source 20 collimated to the volume of interest 59, the volume of change 57 within the volume of interest 59 where changes have occurred could be scanned at a high resolution and the remainder of the volume of interest 59 could be scanned at low resolution. As the x-ray source 20 is collimated, the patient P is exposed to less x-rays. The CT scanner 10 takes a plurality of images at a plurality of angularly-spaced positions while the x-ray source 20 collimated.

In another example, the dataset required to form a multi-resolution image could be taken during a single scan by adapting the x-ray flux spatially (i.e., a low dose for lower resolution volumes and a higher dose for higher resolutions volume). The CT scanner 10 would employ automatic volume of interest 59 localization.

The foregoing description is only exemplary of the principles of the invention. Many modifications and variations are possible in light of the above teachings. It is, therefore, to be understood that within the scope of the appended claims, the invention may be practiced otherwise than using the example embodiments which have been specifically described. For that reason the following claims should be studied to determine the true scope and content of this invention.

What is claimed is:

1. A method of updating a CT scan of a patient, the method comprising the steps of:
performing a partial intra-operative CT scan of a patient to obtain initial images;
comparing the initial images to previous data to automatically determine a volume of change in the patient;
collimating an x-ray source based upon the volume of change to direct x-rays towards at least the volume of change;
performing a collimated intra-operative CT scan of the volume of change to obtain collimated x-ray data; and
reconstructing a CT image based upon the previous data and the collimated x-ray data to create a fully updated CT image.

2. The method as recited in claim 1 further including the step of obtaining the previous data by performing a pre-operative CT scan of the patient and storing the previous data on a computer.

3. The method as recited in claim 1 further including the step of obtaining the previous data from generic data.

4. The method as recited in claim 1 wherein the step of collimating the x-ray source is done with a collimated field of view, and the step of performing the partial intra-operative CT is done with a full field of view that is larger than the collimated field of view.

5. The method as recited in claim 1 wherein a number of the initial images taken during the step of performing the partial intra-operative CT scanner is substantially less than a number of collimated x-ray images taken during the step of performing the collimated intra-operative CT scan.

6. The method as recited in claim 1 wherein the initial images are taken over an angular area of less than 45°

7. The method as recited in claim 1 further including the step of registering a location of the CT scanner relative to the patient and the previous data.

8. The method as recited in claim 1 wherein the step of performing the partial intra-operative CT scan generates partial intra-operative data, and the collimated x-ray data has a higher resolution than the partial intra-operative data and the previous data.

9. The method as recited in claim 1 wherein the step of performing the collimated intra-operative CT scan is done at a higher x-ray dosage than the step of performing the partial intra-operative CT scan.

10. The method as recited in claim 1 further including the step of downsampling data obtained from the partial intra-operative CT scan.

11. The method as recited in claim 1 wherein the step of collimating the x-ray source includes directing the x-rays towards a volume of interest which includes the volume of change and a margin.

12. A CT scanner comprising:
an x-ray source to generate x-rays;
an x-ray detector mounted opposite the x-ray source; and
a computer that stores previous data and compares initial images of a partial intra-operative CT scan to the pre-operative data to define a volume of change in a patient, wherein the x-ray source is then collimated to focus collimated x-rays towards the volume of change to obtain collimated x-ray data of the volume of change, and wherein the computer creates a CT image based upon the previous data and the collimated x-ray data to obtain a fully updated CT image.

13. The CT scanner as recited in claim 12 wherein the x-ray source is a cone-beam x-ray source.

14. The CT scanner as recited in claim 12 further including a gantry including a cross-bar section, a first arm and a second arm that each extend substantially perpendicularly to the cross-bar section, wherein the x-ray source is housed in the first arm and the x-ray detector is housed in the second arm.

15. The CT scanner as recited in claim 12 wherein the previous data is one of generic data and a pre-operative scan of the patient.

16. The CT scanner as recited in claim 12 wherein the previous data and partial intra-operative CT data have a lower resolution than the collimated x-ray data.

17. A method of updating a CT scan of a patient, the method comprising the steps of:
performing a partial intra-operative scan of a patient to obtain initial images at a first resolution;
comparing the initial images to previous data taken at a second resolution to determine a volume of change in the patient;
directing x-rays towards at least the volume of change; and
performing a CT scan of the volume of change to obtain x-ray data at a third resolution, wherein the first resolution and the second resolution are lower than the third resolution.

18. The method as recited in claim 17 further including the steps of updating the previous data with the x-ray data to create a fully updated CT image.

19. The method as recited in claim 17 further including the step of obtaining the previous data by performing a pre-operative scan of the patient and storing the pre-operative scan on a computer.

20. The method as recited in claim 17 wherein the step of performing the partial intra-operative scan is performed at a full field of view.

21. The method as recited in claim 17 further including the step of downsampling data from the initial images.

22. The method as recited in claim 21 wherein the step of downsampling includes sampling pixels of the data from the initial images.

23. The method as recited in claim 21 wherein the step of downsampling includes averaging together a signal from adjacent pixels of the data from the initial images.

24. The method as recited in claim 17 wherein the step of directing the x-rays includes directing the x-rays towards a volume of interest which includes the volume of change and a margin, wherein a resolution of images of the volume of change is greater than a resolution of images of the margin.

25. A method of updating a CT scan of a patient, the method comprising the steps of:
obtaining CT data at a first resolution;
selecting a volume of interest in the CT data;
directing x-rays towards the volume of interest; and
performing a CT scan of the volume of interest to obtain x-ray data at a second resolution, wherein the first resolution is less than the second resolution.

26. The method as recited in claim 25 further including the steps of updating the CT data with the x-ray data to create a fully updated CT image.

27. The method as recited in claim 25 further including the step of obtaining the CT data by performing a pre-operative scan of the patient and storing the pre-operative scan on a computer.

28. The method as recited in claim 25 further including the step of downsampling the CT data.

29. The method as recited in claim 28 wherein the step of downsampling includes sampling pixels of the CT data.

30. The method as recited in claim 28 wherein the step of downsampling includes averaging together a signal from adjacent pixels of the CT data.
